(19) United States
(12) Plant Patent Application Publication
(10) Pub. No.: US 2005/0241041 P1
(43) Pub. Date: Oct. 27, 2005

(54) PETUNIA PLANT NAMED 'DANCASBIPAS'

(50) PETUNIA X HYBRIDA; PETUNIA

(75) Inventor: Gabriel Danziger, Moshav Nir-Zvi (IL)

Correspondence Address: FOLEY AND LARDNER;SUITE 500, 3000 K STREET NW, WASHINGTON, DC, 20007, US

(73) Assignee: Danziger "Dan" Flower Farm

(21) Appl. No.: 10/829,220

(22) Filed: Apr. 22, 2004

(51) Int. Cl. C12Q 1/68; A01H 5/00

(52) U.S. Cl. PLT/356

(57) ABSTRACT

A new and distinct Petunia plant named ‘Dancasbipas’ particularly characterized by its flower color: five white strips over pink background; very floriferous and very long blooming season; vigorous, cascading growth habit; good weather tolerance; and suitable for patios and outdoors bedding.

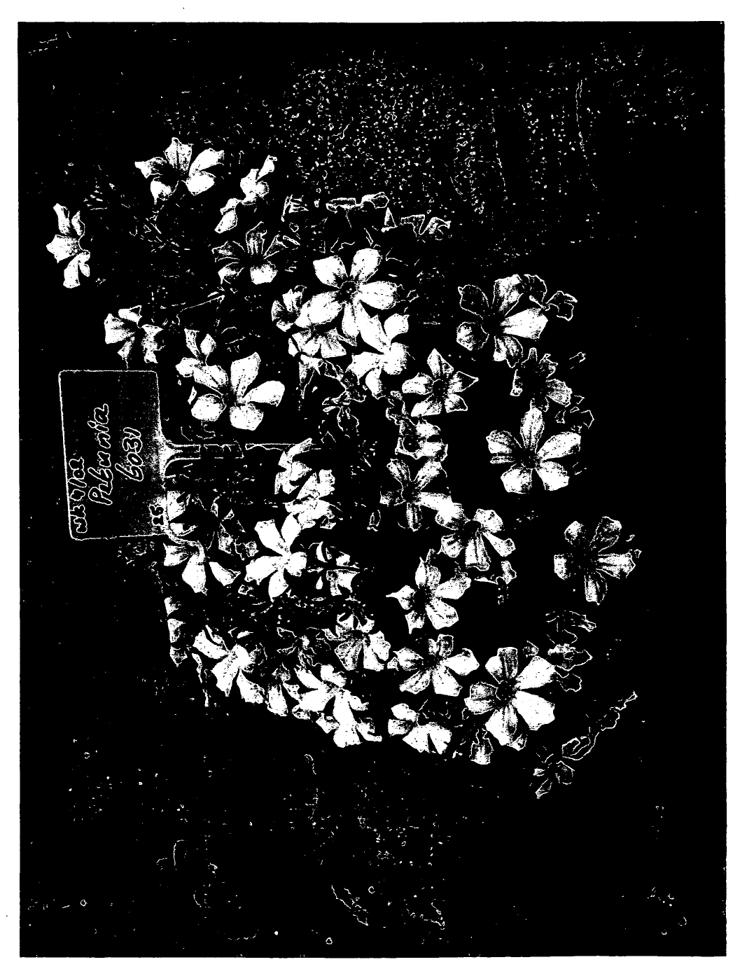

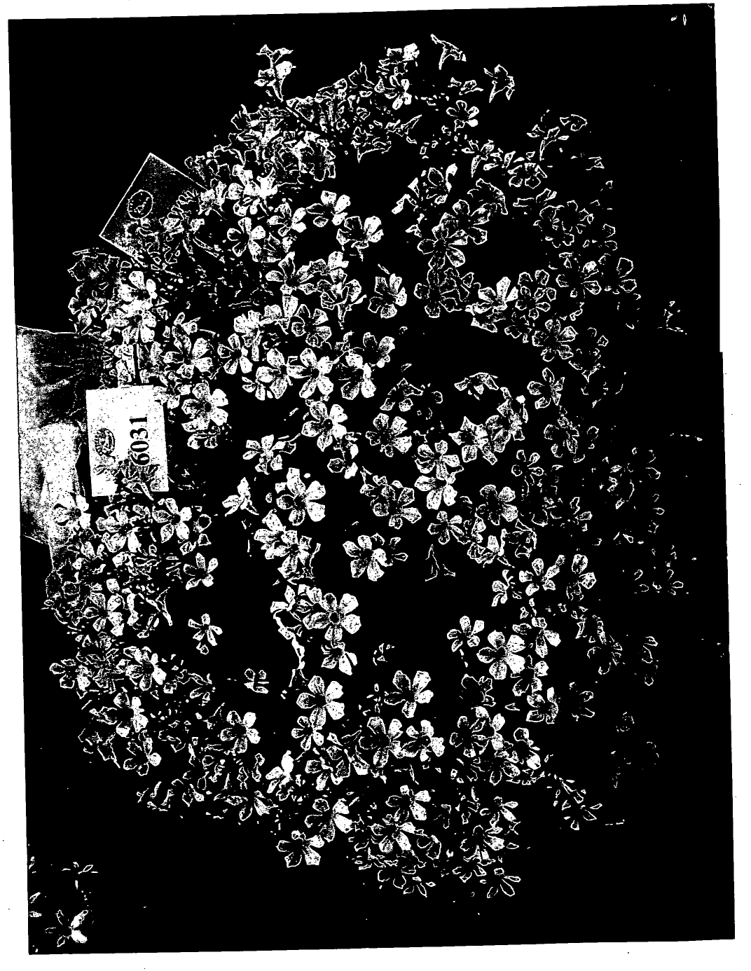

### LATIN NAME OF THE GENUS AND SPECIES OF THE CLAIMED PLANT

Petunia sensu Wijsman (hybrida)

### VARIETY DENOMINATION

Dancasbipas

### BACKGROUND OF THE INVENTION

The present invention relates to a new and distinct cultivar of Petunia plant, botanically known as Petunia sensu Wijsman, hereinafter referred to by the cultivar name ‘Dancasbipas’.

The new cultivar originated from cross made in a controlled breeding program in Moshav Mishmar Hashiva, Israel. The female parent is a proprietary cultivar designated CV-52 (unpatented). The male parent is a proprietary cultivar designated CV-144 (unpatented). ‘Dancasbipas’ was discovered and selected by the inventor, Gabriel Danziger, as a flowering plant within the progeny of the stated cross in a controlled environment in Moshav Mishmar Hashiva, Israel.

Asexual reproduction of the new cultivar by cutting of side shoots, was first performed in February, 2003 in Moshav Mishmar Hashiva, Israel, and has demonstrated that the combination of characteristics as herein disclosed for the new cultivar are firmly fixed and retained through successive generations of asexual reproduction. The new cultivar reproduces true-to-type.

### BRIEF SUMMARY OF THE INVENTION

The following traits have been repeatedly observed and are determined to be basic characteristics of ‘Dancasbipas ’ which in combination distinguish this Petunia as a new and distinct cultivar:

1. Flower color: five white strips over pink background;

2. Very floriferous and very long blooming season;

3. Vigorous, cascading growth habit;

4. Good weather tolerance; and

5. Suitable for patios and outdoors bedding.

‘Dancasbipas ’ has not been observed under all possible environmental conditions. The phenotype of the new cultivar may vary significantly with variations in environment such as temperature, light intensity, and day length without any change in the genotype of the plant. The following observations, measurements and values describe the new cultivar as grown in Moshav Mishmar Hashiva, Israel under conditions, which closely approximate those generally used in commercial practice.

Plants of ‘Dancasbipas’ differ from plants of the parental cultivars, ‘CV-52’ and ‘CV-144’ in the following characteristics:

TABLE 1

| | | Female parent - | Male parent - |
|---|---|---|---|
| Character | ‘Dancasbipas’ | ‘CV-52’ | ‘CV-144’ |
| Flowering | Early, Spring | Late Summer | Mid Summer |
| time: | | | |
| Leaf size: | Medium | Medium | Small |
| Growth | Vigorous, well | Vigorous, well | Medium Vigor and |
| habit: | branched, semi bush | branched | not well branched |

Of the many commercial cultivars known to the present inventor, the most similar in comparison to ‘Dancasbipas ’ is the cultivar ‘Cascadias Bicolor Lavender’ (unpatented).

TABLE 2
Character-

| istic | ‘Dancasbipas’ | Cascadias Bicolor Lavender |
|---|---|---|
| Flowering | Early; spring, 8 weeks | Medium; summer, 10 weeks |
| time | from planting | from planting |
| Leaf size | Medium, length: 7.5 cm | Large, length: 12.5 cm |
| | width: 2.5-3 cm | width: 5 cm |
| Growth habit | Vigorous, well branched, | Medium, cascading, height - |
| | semi bush, height - 25 cm | 15-20 cm |

### BRIEF DESCRIPTION OF THE DRAWING

The accompanying photographic drawings illustrate the overall appearance of the new Petunia showing the colors as true as is reasonably possible with colored reproductions of this type. In the photographs, ‘Dancasbipas’ is labeled by the designation 6031. The first photograph shows a top view of a flowering ‘Dancasbipas’ plant. The second photograph shows a side view of a flowering plant of ‘Dancasbipas’ at 3 months.

### DETAILED BOTANICAL DESCRIPTION

The following observations, measurements and values describe the new cultivar as grown in Moshav Mishmar Hashiva, Israel under conditions which closely approximate those generally used in commercial practice. ‘Dancasbipas’ is for use as a pot plant and a bedding plant. Grown outdoors in moderate climate, indoors during winter. Irrigation and fertilization are needed on a regular basis. For optimal growth, grow in full sunlight ‘Dancasbipas’ plants are fully grown in 3 months. ‘Dancasbipas’ blooms year-round in moderate climate. Color references are made to The Royal Horticultural Society Colour Chart (RHS) 2001 edition, except where general colors of ordinary significance are used. Color values were taken under daylight conditions at approximately noon in Moshav Mishmar Hashiva, Israel. The age of the plant described is 3 months.

### PLANT

General appearance and form:
   Height.—25 cm.
   Spread.—30 inches.
   Growth habit.—Bush cascading.
   Growth rate.—Fully grown in 3 months.
   Branching habit and description.—Freely.
   Flowering response.—Long day facultative.
   Flowering season.—Year round in moderate climate.
   Winter hardiness/weather tolerance.—Frost tender, plants may be damaged in temperatures below 5° C.
   Lastingness of the individual bloom.—2-4 days.
   Rooting habit.—From cutting base.
   Time to initiate roots.—1 week.
   Time to produce a rooted cutting.—2-3 weeks.
   Fragrance.—None.

### STEM DESCRIPTION

Length.—15-20 inches.
Diameter.—5-6 mm.
Internode.—1-1.5 inches in length, color yellow-green RHS 144 A.
Strength.—Medium.
Branching.—Freely.
Pubescence.—Present.

### FOLIAGE

Overall shape of leaf: Elliptic.
Arrangement: Alternate.
Apex: Obtuse.
Base: Acute.
Length: 7.5 cm.
Width: 1-1.25 inches.
Margin: Entire.
Texture: Lustrous.
Color of upper surface:
   Mature leaf.—Yellow-Green RHS 147 A.
   Immature leaf.—Yellow-Green RHS 146 A.
Color of lower surface:
   Mature leaf.—Yellow-Green RHS 146 A.
   Immature leaf.—Yellow-Green RHS 146 A.
Venation color:
   Upper surface.—Yellow-Green RHS 144C.
   Lower surface.—Yellow-Green RHS 144C.
Petiole:
   Length.—1 inch.
   Diameter.—4 mm.
   Color.—Yellow-Green RHS 144C.

### INFLORESCENCE

Flower longevity: 2-4 days.
Flower size: Depth: 2-2.5 cm. Diameter: 4-5 cm.
Flower shape: Trumpet.
Average number of flowers per lateral branch: 6-8.
Floral tube: Diameter: 0.7-1.0 cm.
Petals:
   Number.—5.
   Shape.—Corolla united with 5 lobes and a long straight tube.
   Size.—2 inches in width; 4-5 cm in length from edge of petal to the base of the floral tube.
   Apex.—Truncate.
   Margin.—Entire.
   Texture.—Glabrous.
   Color.—Upper surface: Red-Purple RHS 74A; and white RHS 155A Lower surface: Red-Purple RHS 72C; and white RHS 155A.
Sepals:
   Number.—5.
   Shape.—Elliptic.
   Length.—25 mm.
   Width.—8 mm.
   Apex.—Truncate.
   Margin.—Entire.
   Texture.—Lustrous.
   Color.—Upper surface: Green RHS 137 A Lower surface: Green RHS 137 B.
Bud:
   Rate of opening.—7-10 days.
   Color.—Red-Purple RHS 72C; Green RHS 142 B.
   Shape.—Flanged, composed of 5 petals ruled one around the other creating a cylindrical 5 winged shape.
   Length.—3.5-4 cm.
   Width.—0.5 cm.
   Diameter.—10 mm.
   Peduncle.—40 mm in length; green in color RHS 137 B.

### REPRODUCTIVE ORGANS

Stamen: 5 in number, white RHS 155 A.
Anthers: White RHS 155 A.
Filament: Yellow-Green RHS 145 B.
Stigma: Yellow-Green RHS 146 C.
Ovary: Yellow-Green RHS 146 C.
Pollen: White RHS 155 A.
Seed:
   Number.—40-50.
   Width.—1 mm.
   Length.—1 mm.
   Shape.—Ovoid.
   Color.—Brown RHS 200 C.
Fruit:
   Number.—Few, when naturally pollinated.
   Width.—4 mm.
   Length.—8 mm.
   Shape.—Ellipsoid.
   Color.—Gray-brown RHS 199 A.
Disease/Pest resistance/susceptibility: Unknown.

1. A new and distinct Petunia plant named ‘Dancasbipas’ as illustrated and described herein.
